(19) United States
(12) Patent Application Publication
(10) Pub. No.: US 2022/0127640 A1
(43) Pub. Date: Apr. 28, 2022

(54) ARTIFICIAL MICRORNA PRECURSOR AND IMPROVED MICRORNA EXPRESSION VECTOR CONTAINING THE SAME

(71) Applicant: NATIONAL INSTITUTE OF ADVANCED INDUSTRIAL SCIENCE AND TECHNOLOGY, Tokyo (JP)

(72) Inventors: Masayuki Sano, Tsukuba-shi, Ibaraki (JP); Mahito Nakanishi, Tsukuba-shi, Ibaraki (JP)

(21) Appl. No.: 17/425,193

(22) PCT Filed: Jan. 24, 2020
(86) PCT No.: PCT/JP2020/002519

§ 371 Date: Jul. 22, 2021

(30) Foreign Application Priority Data
Jan. 25, 2019 (JP) 2019-011541

(51) Int. Cl. C12N 15/86; C12N 15/68; C12N 15/113

(52) U.S. Cl. CPC C12N 15/86; C12N 15/68; C12N 15/113; C12N 2310/533; C12N 2310/141; C12N 2310/532; C12N 2760/18843

(57) ABSTRACT

An isolated RNA molecule includes an artificial microRNA precursor comprising in the 5′→3′ direction: a first terminal oligonucleotide consisting of AGGCCR (SEQ ID NO: 1) or a nucleotide sequence in which one to three nucleotides in SEQ ID NO: 1 are substituted; a passenger strand oligonucleotide; a first central oligonucleotide consisting of CYG (SEQ ID NO: 2); a second central oligonucleotide consisting of a nucleotide sequence having at least 70% homology with UUGAAUAKAAAU (SEQ ID NO: 3); a third central oligonucleotide consisting of YGG (SEQ ID NO: 4); a guide strand oligonucleotide; and a second terminal oligonucleotide consisting of UGGAYYK (SEQ ID NO: 5) or a nucleotide sequence in which one to three nucleotides in SEQ ID NO: 5 are substituted.

### RELATED APPLICATIONS

This application is a 35 U.S.C. § 371 national phase application of PCT Application PCT/JP2020/002519, filed Jan. 24, 2020, which claims priority to Japanese Application No. 2019-011541, filed Jan. 25, 2019, which is incorporated herein by reference in its entirety.

### STATEMENT REGARDING ELECTRONIC FILING OF A SEQUENCE LISTING

A Sequence Listing in ASCII text format, submitted under 37 C.F.R. § 1.821, entitled 5576-387_ST25.txt, 7,988 bytes in size, generated on Jul. 22, 2021, and filed via EFS-Web, is provided in lieu of a paper copy. This Sequence Listing is hereby incorporated by reference into the specification for its disclosures.

### TECHNICAL FIELD

The present invention relates to artificial microRNA precursors and to improved microRNA expression vectors containing the same.

### BACKGROUND ART

RNA interference (RNAi) is a phenomenon in which a small double-stranded RNA (siRNA) consisting of about 21 base pairs causes post-transcriptional gene silencing. To supply siRNA stably in cells, a short hairpin RNA (shRNA) expression vector is usually introduced into cells. However, unphysiological overexpression of shRNA is known to cause cytotoxicity by saturating and inhibiting the endogenous microRNA (miRNA) production pathway. To avoid this problem, development of an artificial miRNA expression vector utilizing the stem-loop structure of endogenous miRNA precursors has been attempted, and as such vectors, DNA plasmid vectors and retroviruses are frequently used (Non-Patent Documents 1, 2). However, these vectors intrude into nuclei and express primary transcription products of miRNA, and hence have a risk of causing incorporation into chromosomal DNA of host cells.

On the other hand, the present inventors have developed a cytoplasmic RNA vector based on Sendai virus (SeV), which can express a foreign gene in the cytoplasm with a high efficiency without entering nuclei (Patent Documents 1, 2). This vector allows simultaneous, stable expression of multiple foreign genes from a single vector, and it has no risk of damaging chromosomal DNA of host cells, and thus, has low cytotoxicity. Accordingly, the vector is particularly suitable for production of iPS cells (Patent Document 3) and are currently used for stem cell research, etc., in many laboratories in Japan and abroad. If the vector is useful in the expression of artificial miRNA, it can serve as an excellent tool for gene silencing, and it is expected to contribute to a wide variety of studies including fundamental research and applied research. However, cytoplasmic RNA vectors cannot use the intranuclear miRNA production pathway, and hence suffer from a problem of low miRNA expression efficiency.

### CITATION LIST

### Patent Document

Patent Document 1: WO 2016/114405
Patent Document 2: JP 5633075 B
Patent Document 3: JP 5963309 B

### Non-Patent Document

Non-Patent Document 1: Silva, J. M. et al., Nat. Genet., (2005), Vol. 37, No. 11, pp. 1281-1288
Non-Patent Document 2: Chung, K. H. et al., Nucleic Acids Res., (2006), Vol. 34, No. 7, e53

### SUMMARY OF INVENTION

### Technical Problem

The present invention has been made for the purpose of providing a vector that has low cytotoxicity and is capable of expressing artificial miRNA/siRNA with a high efficiency without adverse influence on host cells.

### Solution to Problem

The present inventors have earnestly researched, and as a result, have succeeded in expressing artificial miRNA/siRNA from various virus vectors or nonviral vectors by using an artificial microRNA precursor based on the structure of an miR-367 precursor.

Specifically, according to one embodiment, the present invention provides an isolated RNA molecule comprising an artificial microRNA precursor comprising in the 5′ 3′ direction: a first terminal oligonucleotide; a passenger strand oligonucleotide; a first central oligonucleotide consisting of CYG (SEQ ID NO: 2), wherein Y is C or U; a second central oligonucleotide consisting of a nucleotide sequence having at least 70% homology with UUGAAUAKAAAU (SEQ ID NO: 3), wherein K is G or U; a third central oligonucleotide consisting of YGG (SEQ ID NO: 4), wherein Y is C or U; a guide strand oligonucleotide; and a second terminal oligonucleotide; wherein the guide strand oligonucleotide consists of 17 to 29 nucleotides having complementarity to a target sequence in an mRNA of a target gene; wherein the passenger strand oligonucleotide has a length identical to the length of the guide strand oligonucleotide or has a length one to three nucleotides shorter than the length of the guide strand oligonucleotide; wherein the first terminal oligonucleotide consists of AGGCCR (SEQ ID NO: 1) or a nucleotide sequence in which one to three nucleotides in SEQ ID NO: 1 are substituted, wherein R is A or G; wherein the second terminal oligonucleotide consists of UGGAYYK (SEQ ID NO: 5) or a nucleotide sequence in which one to three nucleotides in SEQ ID NO: 5 are substituted, wherein Y is C or U independently on each occurrence and K is G or U; wherein the first terminal oligonucleotide and the second terminal oligonucleotide pair to form a first structural stem region; wherein the passenger strand oligonucleotide and the guide strand oligonucleotide pair to form a double-stranded microRNA region; wherein the first central oligonucleotide and the third central oligonucleotide pair to form a second structural stem region; wherein the first structural stem region, the double-stranded microRNA region, and the second structural stem region together form a stem structure; and wherein the second central oligonucleotide forms a loop structure.

The double-stranded microRNA region may include a mismatch or a bulge.

A spacer oligonucleotide consisting of 1 to 10 nucleotides may be further comprised between the first central oligonucleotide and the second central oligonucleotide, or between the second central oligonucleotide and the third central oligonucleotide.

According to one embodiment, the present invention provides an expression vector comprising: the above isolated RNA molecule or an RNA molecule consisting of a complementary sequence thereto, or a DNA molecule coding therefor.

The expression vector is preferably an RNA virus vector, is more preferably a cytoplasmic RNA virus vector, and is particularly preferably a Sendai virus vector.

### Advantageous Effects of Invention

The isolated RNA molecule comprising an artificial microRNA precursor according to the present invention can express artificial miRNA/siRNA with a high efficiency not only from conventional DNA plasmid vectors but also from cytoplasmic RNA virus vectors. Therefore, when used in combination with a cytoplasmic RNA virus vector, the isolated RNA molecule can express artificial miRNA/siRNA with a high efficiency without adverse influence on host cells, thus being useful.

### BRIEF DESCRIPTION OF DRAWINGS

FIG. 1 shows schematic diagrams illustrating the genomic configurations of SeV vectors for expression of miRNA.

FIG. 2 shows a graph representing expression levels of miR-124 in HCT116 cells into which a SeV vector (SeV-124) was introduced.

FIG. 3 shows a graph for evaluation of the gene knockdown effect of miR-124 on the basis of luciferase activity when a reporter gene was introduced into the cells in FIG. 2.

FIG. 4 shows a graph representing expression levels of miR-302a, miR-302b, miR-302c, miR-302d, and miR-367 in HCT116 cells into which a SeV vector (SeV-302-367) was introduced.

FIG. 5 shows a graph for evaluation of the gene knockdown effect of miR-302a on the basis of luciferase activity when a reporter gene was introduced into the cells in FIG. 4.

FIG. 6 shows a graph for evaluation of the gene knockdown effect of miR-367 on the basis of luciferase activity when a reporter gene was introduced into the cells in FIG. 4.

FIG. 7A-FIG. 7E show graphs for comparing expression levels of miR-302a, miR-302b, miR-302c, miR-302d, and miR-367 in HCT116 cells into which an miR-302-367 cluster was introduced by a SeV vector (SeV-302-367) with those expression levels in human iPS cells.

FIG. 8 shows a graph for comparing expression levels of miRNAs in HCT116 cells into which an miR-302-367 cluster was introduced by a retroviral vector (Retro-302-367) and those expression levels in HCT116 cells into which an miR-302-367 cluster was introduced by a SeV vector (SeV-302-367).

FIG. 9 shows a diagram illustrating the secondary structure of an miR-367 precursor (SEQ ID NO: 33).

FIG. 10 shows a graph evaluating the gene knockdown effect of miR-367 expressed from an miR-367 precursor on the basis of luciferase activity.

FIG. 11 shows a diagram illustrating the secondary structure of artificial miR-124 precursor (1) (SEQ ID NO: 20).

FIG. 12 shows a graph evaluating the gene knockdown effect of miR-124 expressed from artificial miR-124 precursor (1) on the basis of luciferase activity.

FIG. 13 shows a diagram illustrating the secondary structure of artificial miR-124 precursor (2) (SEQ ID NO: 21).

FIG. 14 shows a graph evaluating the gene knockdown effect of miR-124 expressed from artificial miR-124 precursor (2) on the basis of luciferase activity.

FIG. 15A-FIG. 15E, show diagrams illustrating the secondary structures of firefly luciferase-targeting artificial miRNA precursors based on various pre-miR structures (FIG. 15A: SEQ ID NO: 22; FIG. 15B: SEQ ID NO: 23; FIG. 15C: SEQ ID NO: 24; FIG. 15D: SEQ ID NO: 25; FIG. 15E: SEQ ID NO: 26).

FIG. 16 shows a graph evaluating the gene knockdown effects of firefly luciferase artificial miRNAs produced from SeV vectors comprising different artificial miRNA precursors shown in FIG. 15A-FIG. 15E on the basis of luciferase activity.

FIG. 17 shows a graph evaluating the gene knockdown effects of firefly luciferase artificial miRNAs produced from CMV plasmid vectors comprising different artificial miRNA precursors shown in FIG. 15A-FIG. 15E on the basis of luciferase activity.

FIG. 18 shows a diagram illustrating the secondary structure of an EGFP-targeting artificial miRNA precursor (pre-miR-367 structure) (SEQ ID NO: 27).

FIG. 19 shows a graph evaluating the gene knockdown effect of an EGFP artificial miRNA produced from a SeV vector comprising the artificial miRNA precursor shown in FIG. 18 on the basis of fluorescence intensity of EGFP.

FIG. 20 shows a diagram illustrating the secondary structure of a mouse p53-targeting artificial miRNA precursor (pre-miR-367 structure) (SEQ ID NO: 28).

FIG. 21 shows a graph evaluating the gene knockdown effect of a mouse p53 artificial miRNA produced from a SeV vector comprising the artificial miRNA precursor shown in FIG. 20 on the basis of luciferase activity.

FIG. 22 shows schematic diagrams illustrating the genomic configurations of a SeV vector expressing reprogramming factors (KLF4, OCT4, SOX2) and a SeV vector expressing reprogramming factors (KLF4, OCT4, SOX2)+p53-targeting artificial miRNA.

FIG. 23 shows a graph evaluating the cell reprogramming efficiency of introduction of each vector shown in FIG. 22 on the basis of expression of SSEA1.

FIG. 24 shows a graph evaluating the gene knockdown effect of p53 artificial miRNA produced from each SeV vector comprising a mouse p53-targeting artificial miRNA precursor on the basis of luciferase activity.

### DESCRIPTION OF EMBODIMENTS

Hereinafter, the present invention will be described in detail; however, the present invention is not limited to embodiments described herein.

According to the first embodiment, the present invention is an isolated RNA molecule comprising an artificial microRNA precursor comprising in the 5′ 3′ direction: a first terminal oligonucleotide; a passenger strand oligonucleotide; a first central oligonucleotide consisting of CYG (SEQ ID NO: 2), wherein Y is C or U; a second central oligonucleotide consisting of a nucleotide sequence having at least 70% homology with UUGAAUAKAAAU (SEQ ID NO: 3), wherein K is G or U; a third central oligonucleotide consisting of YGG (SEQ ID NO: 4), wherein Y is C or U; a guide strand oligonucleotide; and a second terminal oligonucleotide; wherein the guide strand oligonucleotide consists of 17 to 29 nucleotides having complementarity to a target sequence in an mRNA of a target gene; wherein the passenger strand oligonucleotide has a length identical to the length of the guide strand oligonucleotide or has a length one to three nucleotides shorter than the length of the guide strand oligonucleotide; wherein the first terminal oligonucleotide consists of AGGCCR (SEQ ID NO: 1) or a nucleotide sequence in which one to three nucleotides in SEQ ID NO: 1 are substituted, wherein R is A or G; wherein the second terminal oligonucleotide consists of UGGAYYK (SEQ ID NO: 5) or a nucleotide sequence in which one to three nucleotides in SEQ ID NO: 5 are substituted, wherein Y is C or U independently on each occurrence and K is G or U; wherein the first terminal oligonucleotide and the second terminal oligonucleotide pair to form a first structural stem region; wherein the passenger strand oligonucleotide and the guide strand oligonucleotide pair to form a double-stranded microRNA region; wherein the first central oligonucleotide and the third central oligonucleotide pair to form a second structural stem region; wherein the first structural stem region, the double-stranded microRNA region, and the second structural stem region together form a stem structure; and wherein the second central oligonucleotide forms a loop structure.

In the present embodiment, “isolated” means that the RNA molecule according to the present embodiment is in a state purified so as to contain substantially no other nucleic acids; specifically, so that the RNA molecule according to the present embodiment has a purity of at least 90%, preferably at least 95%, and particularly preferably 99% or higher.

In the present embodiment, “artificial microRNA precursor” refers to an unnatural RNA molecule that mimics the structure of a known or wild-type microRNA (hereinafter, also referred to as “miRNA”) precursor and expresses natural or artificial miRNA or siRNA. The scope of the artificial miRNA precursor according to the present embodiment can include both pri-miRNA and pre-miRNA.

The artificial miRNA precursor according to the present embodiment includes, as a first component, a first structural stem region formed by pairing of a first terminal oligonucleotide and a second terminal oligonucleotide. Here, “pair(ing)” means formation of base pairs between two oligonucleotides, and these base pairs may include not only G:C and A:U but also wobble base pairs (G:U). In the present embodiments, the first structural stem region in the artificial miRNA precursor is based on the structures of mouse and human miR-367 precursors, and may be completely identical or substantially the same as the corresponding part of the structure of a mouse or human miR-367 precursor. “Substantially the same” means that nucleotide substitutions are included to a degree that does not affect the entire structure of the stem region (e.g., about one to three nucleotide substitutions).

Specifically, the first terminal oligonucleotide consists of AGGCCR (SEQ ID NO: 1) or a nucleotide sequence in which one to three nucleotides in SEQ ID NO: 1 are substituted, and the second terminal oligonucleotide consists of UGGAYYK (SEQ ID NO: 5) or a nucleotide sequence in which one to three nucleotides in SEQ ID NO: 5 are substituted. Here, R is A or G in the nucleotides of SEQ ID NO: 1, and Y is C or U independently on each occurrence and K is G or U in the nucleotides of SEQ ID NO: 5. Positions and types of nucleotide substitutions are not particularly limited as long as the entire structure of the first structural stem region is retained. Preferably, the first terminal oligonucleotide may consist of AGGCCG (SEQ ID NO: 6) or AGGCCA (SEQ ID NO: 7), and the second terminal oligonucleotide may consist of UGGACCU (SEQ ID NO: 8) or UGGAUUG (SEQ ID NO: 9).

The artificial miRNA precursor according to the present embodiment includes, as a second component, a double-stranded microRNA region formed by pairing of a passenger strand oligonucleotide and the guide strand oligonucleotide. In double-stranded miRNA, “guide strand” refers to a strand that becomes mature miRNA (i.e., an antisense strand in siRNA), and “passenger strand” refers to a strand that is to be removed from double-stranded miRNA and decomposed (i.e., a sense strand in siRNA).

In the present embodiment, the guide strand oligonucleotide consists of 17 to 29 nucleotides, preferably 19 to 25 nucleotides, particularly preferably 21 to 23 nucleotides, and most preferably 22 nucleotides, having complementarity to a target sequence in an mRNA of a target gene. A target sequence in an mRNA of a target gene can be appropriately selected so that expression of the target gene can be specifically suppressed with an already established designing method for artificial miRNA/siRNA in the art.

To completely suppress expression of a target gene, it is preferable that the guide strand oligonucleotide according to the present embodiment consist of a sequence having perfect or complete (i.e., 100%) complementarity to the target sequence; to suppress expression of a target gene to a low to medium degree, it may be sufficient to use a sequence having complementarity to a degree that allows specific recognition of an mRNA of the target gene. Hence, it may be sufficient for the guide strand oligonucleotide according to the present embodiment to have at least 60%, 70%, 80%, 85%, 90%, 95%, 99%, or 100% sequence complementarity to a target sequence in an mRNA of a target gene. In other words, the guide strand oligonucleotide according to the present embodiment may have, for example, a sequence such that 10 or fewer, eight or fewer, six or fewer, five or fewer, four or fewer, three or fewer, two, or one nucleotide substituted in a sequence completely complementary to a target sequence in an mRNA of a target gene. Sequence complementarity can be calculated by using any calculation algorithm conventionally used in the art (e.g., NCBI BLAST).

In the present embodiment, the passenger strand oligonucleotide has a length identical to the length of the guide strand oligonucleotide or has a length one to three nucleotides shorter than the length of the guide strand oligonucleotide. Thus, it follows that if the guide strand oligonucleotide consists of 22 nucleotides, the passenger strand oligonucleotide consists of 19 to 22 nucleotides. In the present embodiment, it is preferable that the passenger strand oligonucleotide have 100% complementarity to the guide strand oligonucleotide; however, the passenger strand oligonucleotide may include, for example, one, two, three, four, or five mismatches or bulges, as long as the passenger strand oligonucleotide and the guide strand oligonucleotide can pair to form a double strand. The position of a mismatch or bulge may be arbitrary, and can be preferably a position corresponding to a mismatch or bulge in mouse and human miR-367 precursors, and specifically the 2-position, the 8-position, and/or the 9-position from the 5′ end of the guide strand oligonucleotide are/is preferred.

The artificial miRNA precursor according to the present embodiment includes, as a third component, a second structural stem region formed by pairing of a first central oligonucleotide consisting of CYG (SEQ ID NO: 2) and a third central oligonucleotide consisting of YGG (SEQ ID NO: 4). Here, Y is C or U independently in each occurrence in the nucleotides of SEQ ID NOs: 2 and 4. In the present embodiment, the second structural stem region in the artificial miRNA precursor is based on the structures of mouse and human miR-367 precursors, and it may be completely identical or substantially the same as the corresponding part of the structure of a mouse or human miR-367 precursor. Preferably, the first central oligonucleotide may consist of CUG (SEQ ID NO: 10), and the third central oligonucleotide may consist of UGG (SEQ ID NO: 11).

In the artificial miRNA precursor according to the present embodiment, the first structural stem region, the double-stranded miRNA region, and the second structural stem region together form a stem structure. Here, the stem structure may consist only of the first structural stem region, the double-stranded miRNA region, and the second structural stem region, or may include nucleotide insertions between the first structural stem region and the double-stranded miRNA region and/or between the double-stranded miRNA region and the second structural stem region to a degree that does not affect the entire stem structure. Specifically, a few (e.g., one, two, or three) nucleotide insertions may exist between the first terminal oligonucleotide and the passenger strand oligonucleotide, between the passenger strand oligonucleotide and the first central oligonucleotide, between the third central oligonucleotide and the guide strand oligonucleotide, and/or between the guide strand oligonucleotide and the second terminal oligonucleotide. In the present embodiment, it is preferable that the stem structure of the artificial miRNA precursor be composed only of the first structural stem region, the double-stranded miRNA region, and the second structural stem region directly linked together.

The artificial miRNA precursor according to the present embodiment includes, as a fourth component, a second central oligonucleotide consisting of a nucleotide sequence having at least 70% homology with UUGAAUAKAAAU (SEQ ID NO: 3). Here, K is G or U in the nucleotides of SEQ ID NO: 3. The second central oligonucleotide according to the present embodiment is based on the structures of mouse and human miR-367 precursors, and may be in any mode that allows formation of a loop structure similar to those of mouse and human miR-367 precursors. Thus, the second central oligonucleotide according to the present embodiment may consist of a nucleotide sequence having at least 70% or 80% homology with a nucleotide sequence consisting of UUGAAUAKAAAU (SEQ ID NO: 3), and may consist of a nucleotide sequence preferably having at least 90% or higher homology, particularly preferably having 100% homology, with SEQ ID NO: 3. In other words, the second central oligonucleotide according to the present embodiment may have a nucleotide sequence such that, for example, four or fewer, three or fewer, two, or one nucleotide is substituted, deleted, or inserted in a nucleotide sequence consisting of UUGAAUAKAAAU (SEQ ID NO: 3). Preferably, the second central oligonucleotide may consist of UUGAAUAGAAAU (SEQ ID NO: 12) or UUGAAUAUAAAU (SEQ ID NO: 13).

In the present embodiment, it is preferable that the first central oligonucleotide, the second central oligonucleotide, and the third central oligonucleotide be directly linked together; however, a spacer oligonucleotide consisting of 1 to 10 nucleotides, one to five nucleotides, or one to three nucleotides may be included between the first central oligonucleotide and the second central oligonucleotide, or between the second central oligonucleotide and the third central oligonucleotide. The spacer oligonucleotide may have any sequence, but it is preferable for the spacer oligonucleotide to have a sequence that does not form base pairs with the nucleotide sequence consisting of UUGAAUAKAAAU (SEQ ID NO: 3).

The isolated RNA molecule according to the present embodiment may be prepared with optional addition of a flanking sequence to the 5′ end and/or 3′ end of an artificial miRNA precursor designed according to the above description. Such a flanking sequence can be appropriately determined according to an expression vector into which the isolated RNA molecule is incorporated. The flanking sequence may include a sequence corresponding to the flanking sequence of a natural miR-367 precursor, and the length may be, for example, 1 to 100 nucleotides, 1 to 50 nucleotides, or 1 to 40 nucleotides, and may be preferably 15 to 25 nucleotides.

The isolated RNA molecule according to the present embodiment can be chemically synthesized or biosynthesized through a gene engineering procedure by using a method known in the art. For example, the isolated RNA molecule according to the present embodiment can be produced through preparation of template DNA followed by transcription thereof with RNA polymerase. The isolated RNA molecule according to the present embodiment may be composed totally of RNA or contain in part modified RNA. Examples of modified RNA include phosphorothioated RNA, boranophosphated RNA, 2′-O-methylated RNA, 2′-F RNA, and 2′,4′-BNA (also known as: LNA (Locked Nucleic Acid)).

Artificial miRNA/siRNA can be expressed by introducing the isolated RNA molecule according to the present embodiment or a DNA molecule encoding thereof into cells. Introduction of the RNA molecule or the DNA molecule into cells can be performed by using a well-known method in the art according to the type of cell, for example, by lipofection, microinjection, or electroporation.

Alternatively, artificial miRNA/siRNA can be expressed with a high efficiency by incorporating the isolated RNA molecule according to the present embodiment into various expression vectors, including cytoplasmic RNA virus vectors, and then introducing the result into a cell.

Hence, according to the second embodiment, the present invention is an expression vector comprising the above isolated RNA molecule, an RNA molecule consisting of a complementary sequence thereto, or a DNA molecule encoding the isolated RNA molecule or the RNA molecule.

The expression vector applicable in the present embodiment is not limited to particular types, and both virus vectors and nonviral vectors can be used. Examples of virus vectors include, but are not limited to, DNA virus vectors such as adenovirus vectors, adeno-associated virus vectors, and herpesvirus vectors, and RNA virus vectors such as retrovirus vectors, lentivirus vectors, bornavirus vectors, and paramyxovirus vectors. Examples of nonviral vectors include plasmid vectors such as pOL1 (produced in Examples below), pCI Mammalian Expression Vector (Promega Corporation), and pBApo-CMV DNA (Takara Bio Inc.), and episomal vectors such as pEBMulti-Hyg (FUJIFILM Corporation).

The expression vector according to the present embodiment is preferably an RNA virus vector, and more preferably a cytoplasmic RNA virus vector. The cytoplasmic RNA virus vector may be selected from, for example, paramyxovirus vectors such as Sendai virus vectors, alphavirus vectors such as Sindbis virus vectors, flavivirus vectors such as tick-borne encephalitis virus vectors, and vesiculovirus vectors such as vesicular stomatitis virus vectors. The expression vector according to the present embodiment can be particularly preferably a Sendai virus vector.

The expression vector according to the present embodiment can be prepared by operably linking the above isolated RNA molecule or an RNA molecule consisting of a complementary sequence thereto, or a DNA molecule encoding the isolated RNA molecule or the RNA molecule to the downstream of a promoter in an expression vector. One or more isolated RNA molecules, or DNA molecules coding therefor as defined above, may be introduced into a single expression vector. In the case in which the expression vector is a Sendai virus vector, for example, it is suitable to insert the above isolated RNA molecule between a gene-start signal and a gene-end signal, and 1 to 10, 1 to 5, 1 to 3, 2, or 1 isolated RNA molecule(s) as defined above inserted between a gene-start signal and a gene-end signal can be introduced into a single Sendai virus vector.

Introduction of the expression vector according to the present embodiment into cells can be performed by using a well-known method in the art according to the type of cells and expression vectors. For nonviral vectors, for example, introduction can be performed with lipofection, electroporation, or microinjection. For virus vectors, introduction can be performed by infecting cells with an appropriate titer or multiplicity of infection (MOI).

The isolated RNA molecule according to the first embodiment and the expression vector according to the second embodiment can express artificial miRNA/siRNA with a significantly higher efficiency than conventional artificial miRNA/siRNA expression systems, thus being useful.

### EXAMPLES

The present invention will be further described with reference to Examples shown below. These by no means limit the present invention.

1. Expression of Various miRNAs from Sendai Virus Vector
(1-1) Production of miRNA Expression Vector

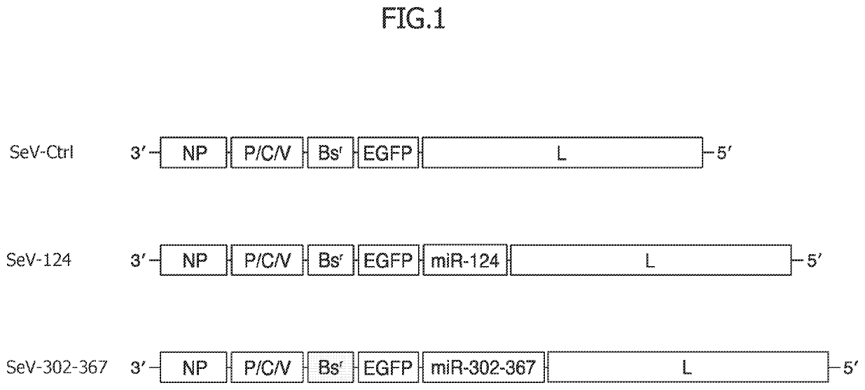

Various natural miRNA precursors were introduced into Sendai virus (SeV) vectors, and expression levels of miRNAs and gene knockdown effects were evaluated. The SeV vector used was a SeVdp vector (J. Biol. Chem., (2011), Vol. 286, No. 6, pp. 4760-4771). To the downstream of the P/C/V in the SeVdp vector, a selection marker blasticidin resistance gene (blasticidin S deaminase gene; prepared by PCR using a pCX4bsr plasmid (Proc. Natl. Acad. Sci. USA, (2003), Vol. 100, No. 23, pp. 13567-13572) as a template) and an expression marker EGFP gene (prepared by PCR using a pEGFP-1 plasmid (Takara Bio Inc.) as a template) were introduced, and an miR-124 gene or an miR-302-367 cluster was further introduced downstream thereto to prepare a miR-124 expression vector (SeV-124) and a miR-302-367 expression vector (SeV-302-367). The miR-124 gene and miR-302-367 cluster had been prepared by PCR using, as a template, a genomic DNA extracted from C57BL/6J mouse embryonic fibroblasts (MEF). A vector containing no miRNA gene (SeV-Ctrl) was prepared as a negative control. FIG. 1 shows the genomic configurations of SeV-124, SeV-302-367, and SeV-Ctrl.

In addition, a retrovirus vector into which the miR-302-367 cluster had been introduced (Retro-302-367) was prepared in the following procedure. The miR-302-367 cluster cloned from MEF was introduced into the BamHI and NotI sites of a pCX4pur plasmid (Proc. Natl. Acad. Sci. USA, (2003), Vol. 100, No. 23, pp. 13567-13572). HEK293T cells were transfected with the plasmid vector obtained together with pVPack-GP (Agilent Technologies) and pVPack-Ampho (Agilent Technologies) by using FuGENE HD (Promega Corporation). On day 3, the culture supernatant was collected and filtered through a 0.45-μm filter to prepare a miR-302-367 expression retrovirus vector.

(1-2) Quantification of Expression Levels of miRNA

HCT116 cells were infected with the SeV vector at MOI=5, cultured from the next day with supplementing the medium with 10 μg/ml blasticidin S, and cells stably retaining the SeV vector genome were selected. For the retrovirus vector, HCT116 cells were infected with 1×10⁹ copies of the vector in the presence of 4 μg/ml polybrene, cultured with supplementing the medium with 0.2 μg/ml puromycin on day 3, and cells stably expressing the miRNA introduced were selected. From the cells, total RNA was extracted by using the ISOGEN reagent (NIPPON GENE CO., LTD.), and RT-qPCR was carried out for each miRNA by using TaqMan MicroRNA Assays (Applied Biosystems). A TaqMan MicroRNA Reverse Transcription Kit (Applied Biosystems) was used for the RT reaction, and TaqMan Universal PCR Master Mix II, no UNG (Applied Biosystems), was used for qPCR. The expression level of each miRNA was evaluated by using a ΔΔCt method in which the Cq (quantification cycle) value for each miRNA was normalized with the Cq value for RNU48 (endogenous control gene). The TaqMan MicroRNA Assays used for RT-qPCR are shown in the following.

TABLE 1
TaqMan MicroRNA Assays for RT-qPCR

| Name of miRNA | Assay ID |
|---|---|
| miR-124 | 001182 |
| miR-302a | 000529 |
| miR-302b | 000531 |
| miR-302c | 002558 |
| miR-302d | 000535 |
| miR-367 | 000555 |
| RNU48 | 001006 |

(1-3) Evaluation of Gene Knockdown Effect of miRNA

To evaluate the gene knockdown effect of each miRNA, a reporter vector was prepared by introducing a target sequence having complete complementarity to a miRNA into the 3′ untranslated region of the RLuc gene in the psiCHECK-2 vector (Promega Corporation) comprising a firefly luciferase (FLuc) gene and a Renilla luciferase (RLuc) gene. FLuc and RLuc are expressed from this reporter vector, and when gene knockdown is caused by a miRNA, only the expression of RLuc decreases. Therefore, the activity of RLuc corrected for the transfection efficiency of the reporter vector can be measured by calculating the relative values of RLuc/FLuc.

Cells prepared in (1-2) were transfected with the above reporter vector by using the Lipofectamine 2000 reagent (Thermo Fisher Scientific). About 22 to 25 hours thereafter, the activities of FLuc and RLuc were measured by using a Dual-Luciferase Reporter Assay System (Promega Corporation), and relative values of the RLuc activity (hereinafter, expressed as “RLuc/FLuc value”) were calculated. As a control, a vector incorporating a scramble sequence which is not targeted by a miRNA, in place of a target sequence for a miRNA, was prepared, and cells were transfected therewith to prepare negative control cells, for which luciferase activities were evaluated in the same manner. Scramble sequences were designed by using siRNA Wizard v3.1 Software (InvivoGen). The gene knockdown effect of each miRNA was evaluated based on the activity of a reporter luciferase RLuc by calculating the relative value of RLuc/FLuc in reporter vector-transfected cells when the RLuc/FLuc value in negative control cells was set to 1.0. The target sequences for miRNAs and the corresponding scramble sequences are shown in the following.

TABLE 2
Target sequences for miRNAs and corresponding
scramble sequences

| Name of target | | SEQ |
|---|---|---|
| sequence | Nucleotide sequence | ID NO |
| 124-T | GGCATTCACCGCGTGCCTTA | 14 |
| 302a-T | TCACCAAAACATGGAAGCACTTA | 15 |
| 367-T | TCACCATTGCTAAAGTGCAATT | 16 |
| Name of scramble | | SEQ |
| sequence | Nucleotide sequence | ID NO |
| 124-scrambleT | GCGAGGTTCCCCTGTCTACA | 17 |
| 302a-scrambleT | GCAGTACACCACGTAAATCAAAT | 18 |
| 367-scrambleT | GCATTCATAATTCTCGGAACTA | 19 |

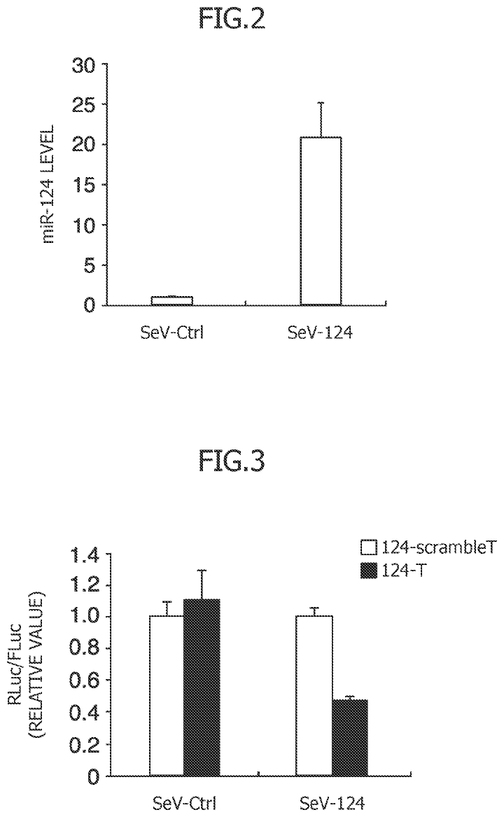

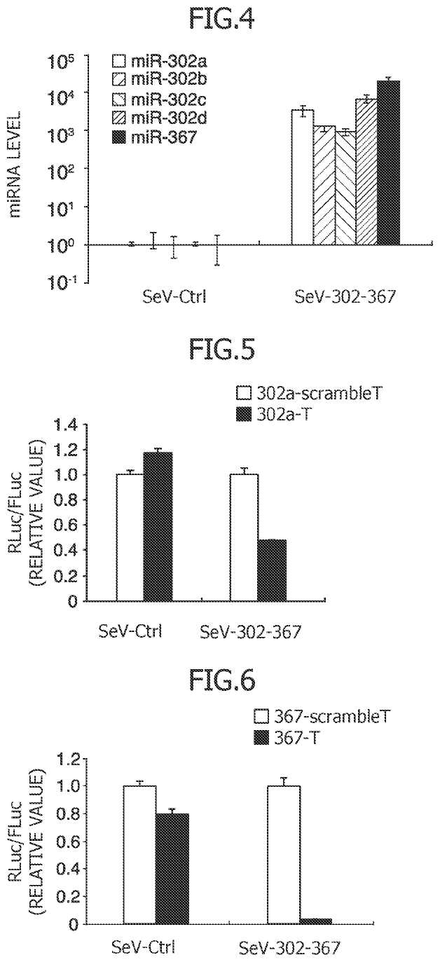

The results are shown in FIGS. 2 to 6. Introduction of SeV-124 resulted in increase in the expression level of miR-124 in HCT116 cells by about 20 times (FIG. 2), and about 53% of RLuc activity was suppressed (FIG. 3). Introduction of SeV-302-367 resulted in the increase of the expression levels of miR-302a, miR-302b, miR-302c, miR-302d, and miR-367 in HCT116 cells by about 900 to 20000 times (FIG. 4), and for example, miR-302a suppressed RLuc activity to about 52% (FIG. 5). In particular, miR-367 exhibited a high target gene knockdown effect, suppressing about 96% of RLuc activity (FIG. 6).

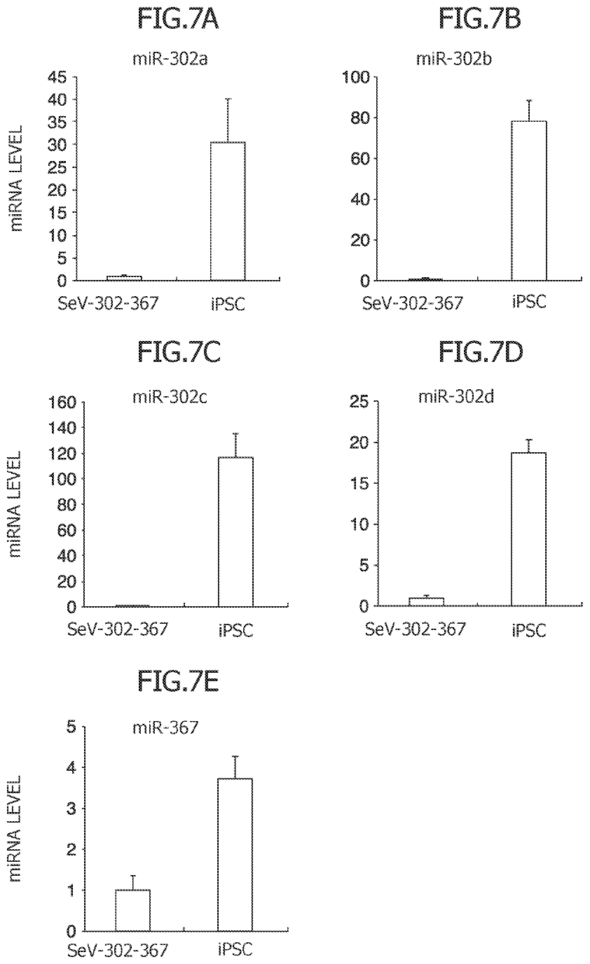

Expression levels of miR-302a, miR-302b, miR-302c, miR-302d, and miR-367 in human iPS cells (PLOS ONE, (2016), Vol. 11, No. 10, e0164720) were quantified in the same procedure as in (1-2), and compared with those in HCT116 cells into which SeV-302-367 had been introduced. The results are shown in FIG. 7A-FIG. 7E. The expression levels of miR-302a, miR-302b, miR-302c, and miR-302d in the SeV-302-367-introduced HCT116 cells were extremely lower than those in the iPS cells, whereas no very large difference was found between both cells for the expression levels of miR-367.

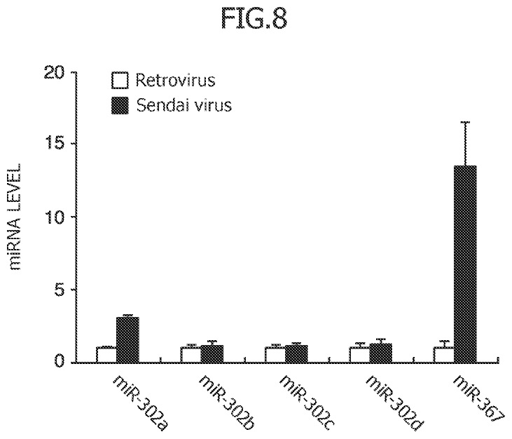

Furthermore, the expression levels of miR-302a, miR-302b, miR-302c, miR-302d, and miR-367 in the Retro-302-367-introduced HCT116 cells and those in the SeV-302-367-introduced HCT116 cells were compared. The results are shown in FIG. 8. While no large difference was found between the expression levels of miR-302a, miR-302b, miR-302c, and miR-302d for Retro-302-367 and those for SeV-302-367, the expression level of miR-367 was significantly higher in the SeV-302-367-introduced cells.

These results suggested the possibility that the expression efficiency of miR-367 from the SeV vector was particularly high.

2. Gene Knockdown Effect of miR-367 Expressed from SeV-367

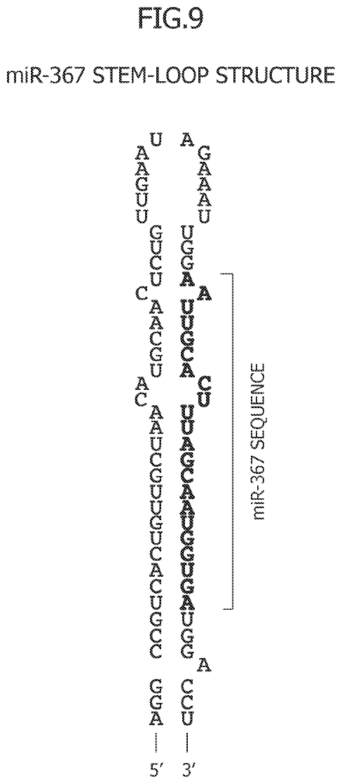

A SeV expression vector into which only an miR-367 precursor (FIG. 9) had been introduced (SeV-367), in place of the miR-302-367 cluster, was prepared in the same procedure as in (1-1), the expression vector was introduced into HCT116 cells in the same procedure as in (1-2), and the gene knockdown effect was evaluated in the same procedure as in (1-3).

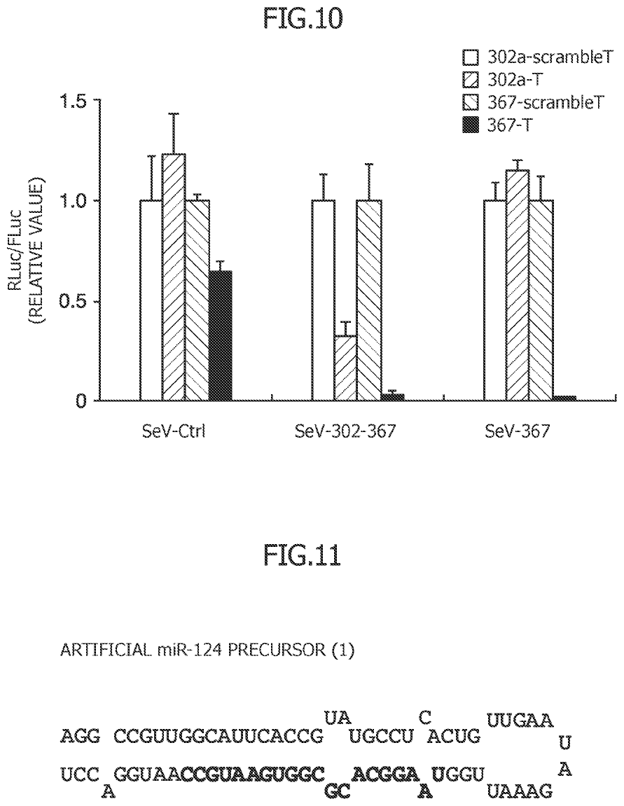

The results are shown in FIG. 10. As with the case of the SeV-302-367-introduced HCT116 cells, a very high target gene knockdown effect was found in the SeV-367-introduced HCT116 cells. These results demonstrated that miR-367 can be expressed at a high level even by a SeV vector into which only the miR-367 precursor has been incorporated.

3. Expression of miR-124 from artificial miR-124 precursor based on miR-367 precursor Artificial miR-124 precursors (1) and (2) were designed as artificial miRNA precursors based on the secondary structure of the miR-367 precursor: artificial miR-124 precursor (1) was obtained by replacing the miR-367 sequence with an miR-124 sequence while the secondary structure of the miR-367 precursor was completely retained, and artificial miR-124 precursor (2) was obtained by further modifying artificial miR-124 precursor (1) not to include any mismatch/bulge in the double-stranded miR region while the structure of the miR-367 precursor was retained. The nucleotide sequences of artificial miR-124 precursors (1) and (2) are shown in Table 3, and the secondary structures are shown in FIG. 11 and FIG. 13. In the figures, the miR-124 sequence is shown in bold. The secondary structures were those predicted by using an mfold web server (Nucleic Acids Res., (2003), Vol. 31, No. 13, pp. 3406-3415).

TABLE 3
Nucleotide sequences of artificial
miR-124 precursors

| Name of | | SEQ |
|---|---|---|
| artificial | | ID |
| miR | Nucleotide sequence | NO |
| Artificial | AGGCCGUUGGCAUUCACCGUAUGCCUCACU | 20 |
| miR-124 | GUUGAAUAGAAAUUGGUAAGGCACGCGGUG | |
| precursor (1) | AAUGCCAAUGGACCU | |
| Artificial | AGGCCGUUGGCAUUCACCGCGUGCCUUACU | 21 |
| miR-124 | GUUGAAUAGAAAUUGGUAAGGCACGCGGUG | |
| precursor (2) | AAUGCCAAUGGACCU | |

A SeV expression vector into which artificial miR-124 precursor (1) or artificial miR-124 precursor (2) had been incorporated was prepared using the same procedure as in (1-1), the expression vector was introduced into HCT116 cells in the same procedure as in (1-2), and the gene knockdown effects were evaluated in the same procedure as in (1-3).

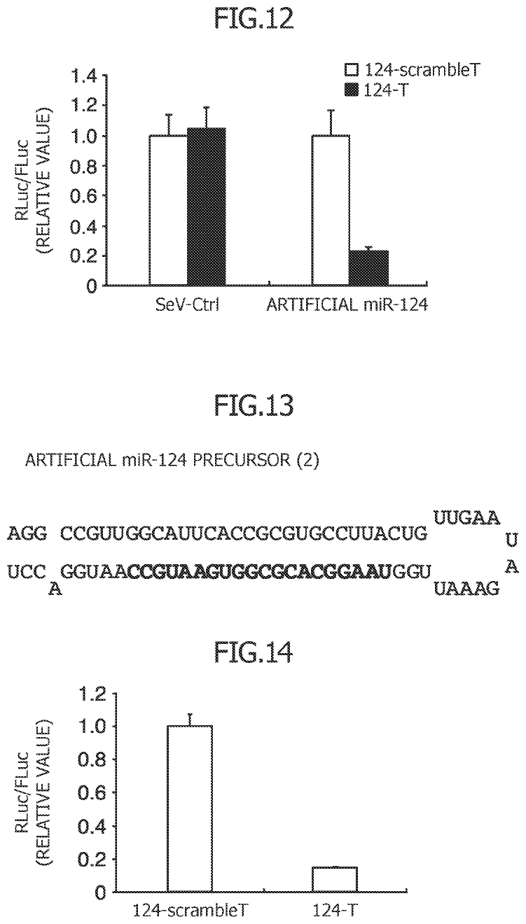

FIG. 12 shows the results for artificial miR-124 precursor (1), and FIG. 14 shows the results for artificial miR-124 precursor (2). Artificial miR-124 precursor (1) suppressed about 77% of RLuc activity, and artificial miR-124 precursor (2) suppressed about 86% of RLuc activity. These results confirmed that different types of miRNAs with high activity can be expressed by using the miR-367 precursor.

4. Target Gene Knockdown Effect of Artificial miRNA Expressed by Artificial miRNA Precursor Based on Pre-miR-367 (1)

Artificial miRNA precursors targeting the FLuc gene were produced based on the structures of various natural miRNA precursors in a procedure shown below, and the gene knockdown effects of FLuc-targeting artificial miRNAs expressed thereby were compared.

(4-1) Expression of Artificial miRNA from SeV Vector

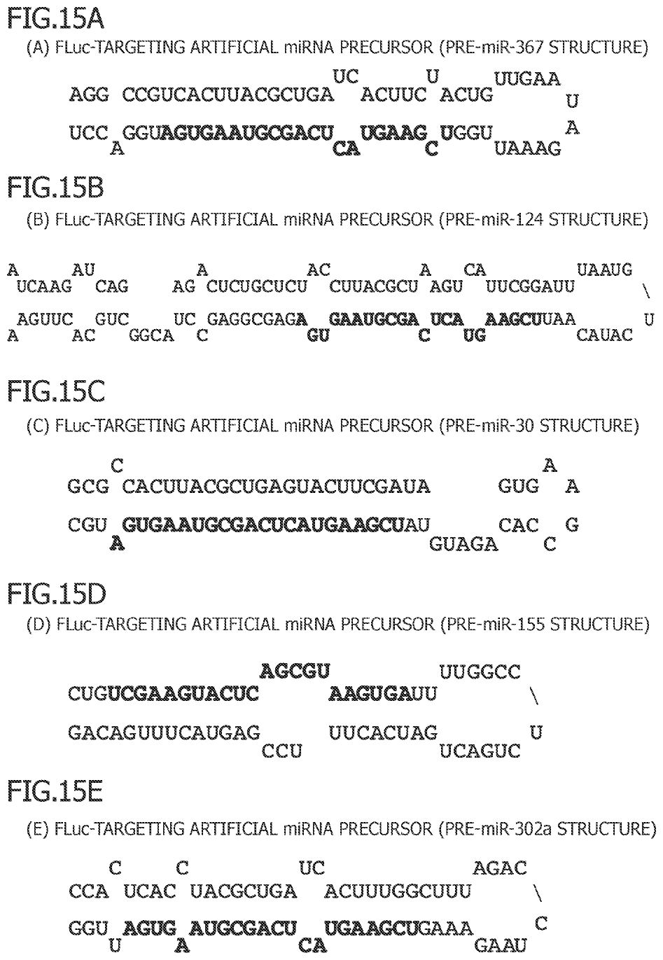

FLuc-targeting artificial miRNA precursors mimicking their secondary structures were designed based on the structures of a miRNA precursor described in Non-Patent Document 1 (pre-miR-30), a miRNA precursor described in Non-Patent Document 2 (pre-miR-155), and mouse-derived natural miRNA precursors (pre-miR-367, pre-miR-124, and pre-miR-302a). A sequence completely complementary to a target sequence in an mRNA for FLuc, described by Elbashir et al. (Nature, (2001), Vol. 411, No. 6836, pp. 494-498), was used for the sequence of each artificial miRNA. The nucleotide sequences of the FLuc-targeting artificial miRNA precursors are shown in Table 4, and the secondary structures are shown in FIG. 15A-FIG. 15E. In the figure, miRNA sequences targeting the FLuc gene are shown in bold.

TABLE 4
Nucleotide sequences of precursors

| Name of artificial miR | Nucleotide sequence | SEQ ID NO |
|---|---|---|
| FLuc-targeting artificial miRNA | AGGCCGUCACUUACGCUGAUCACUUCUACU | 22 |
| precursor (pie-mi R-367 structure) | GUUGAAUAGAAAUUGGUCGAAGUACUCAGC | |
| | GUAAGUGAUGGACCU | |
| FLuc-targeting artificial miRNA | AUCAAGAUCAGAGACUCUGCUCUACCUUAC | 23 |
| precursor (pre-miR-124 structure) | GCUAAGUCAUUCGGAUUUAAUGUCAUACAA | |
| | UUCGAAGUACUCAGCGUAAGUGAGAGCGGA | |
| | GCCUACGGCUGCACUUGAA | |
| FLuc-targeting artificial miRNA | GCGCCACUUACGCUGAGUACUUCGAUAGUG | 24 |
| precursor (pre-mi R-30 structure) | AAGCCACAGAUGUAUCGAAGUACUCAGCGU | |
| | AAGUGAUGC | |
| FLuc-targeting artificial miRNA | CUGUCGAAGUACUCAGCGUAAGUGAUUUUG | 25 |
| precursor (pre-miR-155 structure) | GCCUCUGACUGAUCACUUUCCGAGUACUUU | |
| | GACAG | |
| FLuc-targeting artificial miRNA | CCACUCACCUACGCUGAUCACUUUGGCUUU | |
| precursor (pre-miR-302a structure) | AGACCUAAGAAAGUCGAAGUACUCAGCGUA | 26 |
| | AGUGAUUGG | |

SeV expression vectors, each incorporating any of the artificial miRNA precursors, were prepared using the same procedure as in (1-1), each expression vector was introduced into HCT116 cells using the same procedure as in (1-2), and selection was then carried out with blasticidin S. Furthermore, a pGL3-Control vector (Promega Corporation) comprising a sequence encoding FLuc, and a pRL-TK vector (Promega Corporation) comprising a sequence encoding RLuc were introduced into the cells by using the Lipofectamine 2000 reagent, and activities of FLuc and RLuc were measured about 24 hours thereafter, and relative values of FLuc activity (hereinafter, expressed as “FLuc/RLuc value”) were calculated. The gene knockdown effect of each artificial miRNA was evaluated by calculating the relative value of FLuc/RLuc in the cells introduced with the SeV expression vector incorporating each artificial miRNA when the FLuc/RLuc value was set to 1.0 in cells prepared in the same manner, except that SeV-Ctrl was used in place of a SeV expression vector incorporating the artificial miRNA precursor.

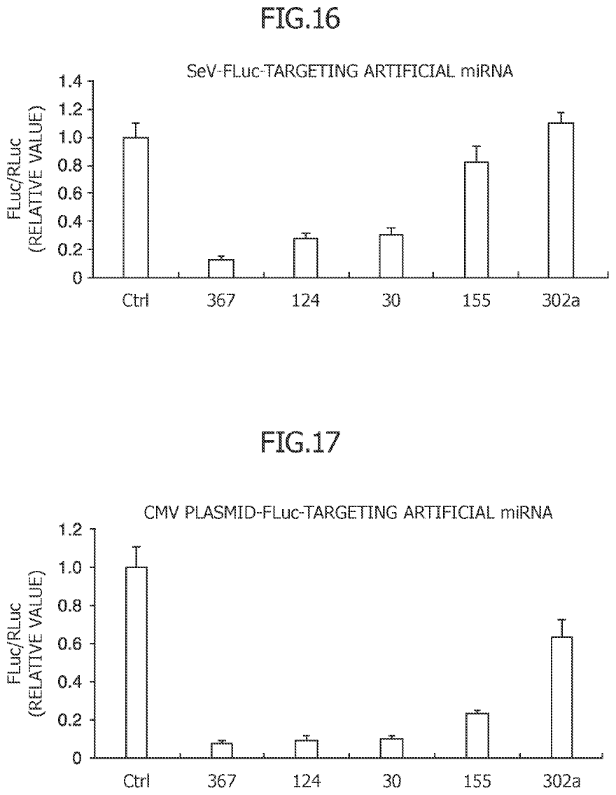

The results are shown in FIG. 16. The artificial miRNA expressed from a SeV vector incorporating the pre-miR-367-based artificial miRNA precursor was found to exhibit the highest gene knockdown effect.

(4-2) Expression of Artificial miRNA by Plasmid Vector

With use of a pOL1 plasmid in place of a SeV vector, plasmid vectors, each incorporating any of the artificial miRNA precursors shown in FIG. 15A-FIG. 15E, downstream of a cytomegalovirus (CMV) promoter of the pOL1 plasmid, were prepared. pOL1 had been produced by substituting a Zeocin resistance gene of pON1 (PLOS ONE, (2016), Vol. 11, No. 10, e0164720) with a neomycin resistance gene. Each CMV plasmid vector incorporated an artificial miRNA precursor, a pGL3-Control vector and a pRL-TK vector were introduced into HCT116 cells by using the Lipofectamine 2000 reagent. Activities of FLuc and RLuc were measured about 24 hours thereafter, and the FLuc/RLuc values were calculated. The gene knockdown effect of each artificial miRNA was evaluated by calculating the relative value of FLuc/RLuc in cells into which a plasmid vector incorporating an artificial miRNA had been introduced were calculated when the FLuc/RLuc value was set to 1.0 in cells (negative control) prepared in the same manner, except that a CMV plasmid vector containing no artificial miRNA precursor was used in place of a CMV plasmid vector incorporating an artificial miRNA precursor.

The results are shown in FIG. 17. Even for the case of the CMV plasmid vector, the artificial miRNA expressed from the pre-miR-367-based artificial miRNA precursor was found to exhibit the highest gene knockdown effect. These results demonstrated that an artificial miRNA can be expressed at a high level, irrespective of the type of expression vector, by using the pre-miR-367-based artificial miRNA precursor, and a high gene knockdown effect can be obtained.

5. Target Gene Knockdown Effect of Artificial miRNA Expressed by Artificial miRNA Precursor Based on Pre-miR-367 (2)

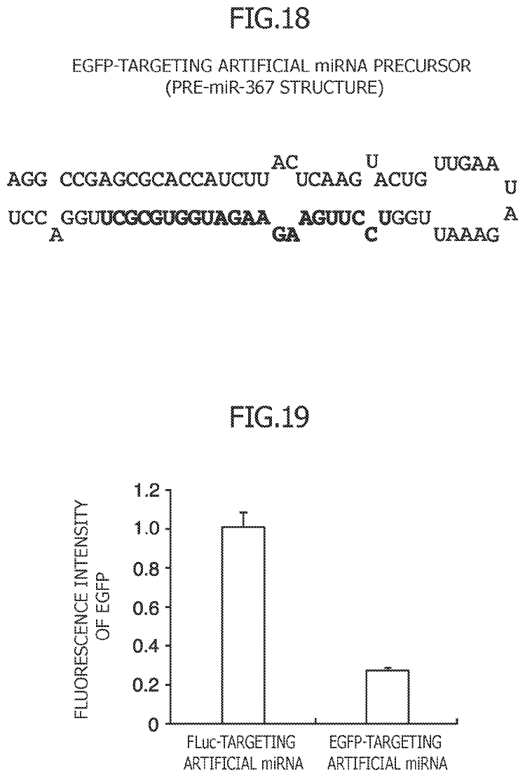

On the basis of the structure of pre-miR-367, an artificial miRNA precursor mimicking the secondary structure thereof and targeting EGFP was produced, and the gene knockdown effect of the EGFP-targeting artificial miRNA expressed from a SeV vector incorporating the artificial miRNA precursor was evaluated. A sequence completely complementary to a target sequence in an mRNA for EGFP (NCBI: Pr008808666) was used for the sequence of the artificial miRNA. The nucleotide sequence of the EGFP-targeting artificial miRNA precursor is shown in Table 5, and the secondary structure is shown in FIG. 18. In the figure, the miRNA sequence targeting the EGFP gene is shown in bold.

TABLE 5
Nucleotide sequence of precursor

| Name of artificial miR | Nucleotide sequence | SEQ ID NO |
|---|---|---|
| EGFP-targeting artificial miRNA | AGGCCGAGCGCACCAUCUUACUCAAGUACU | 27 |
| precursor (pre-miR-367 structure) | GUUGAAUAGAAAUUGGUCCUUGAAGAAGAU | |
| | GGUGCGCUUGGACCU | |

A SeV vector incorporating the EGFP-targeting artificial miRNA precursor, a hygromycin resistance gene (hygromycin B phosphotransferase gene, obtained by artificial gene synthesis (GenScript Biotech)), as a selection marker, and a Keima-Red gene (prepared by PCR using a phdKeima-Red-S1 plasmid (Medical & Biological Laboratories Co., Ltd.) as a template), as an expression marker, was prepared using the same procedure as in (1-1). The expression vector was introduced into HCT116 cells using the same procedure as in (1-2), the medium was supplemented with 100 μg/ml hygromycin B from the next day, and cells stably retaining the SeV vector genome were selected. Into the cells obtained, a pEGFP-N1 plasmid (Clontech Laboratories, Inc.) and an E2-Crimson expression plasmid were introduced by using the Lipofectamine 2000 reagent. The next day, fluorescence intensities of EGFP and E2-Crimson were measured by flow cytometry. In addition, measurement of fluorescence intensities was performed in the same manner, except that a SeV expression vector incorporating an FLuc-targeting artificial miRNA precursor was used in place of a SeV expression vector incorporating the EGFP-targeting artificial miRNA precursor (negative control). The gene knockdown effect of EGFP-targeting artificial miRNAs was evaluated by calculating the relative value of the fluorescence intensity of EGFP in E2-Crimson positive cells in the negative control as 1.0. The E2-Crimson expression plasmid had been produced by incorporating an E2-Crimson gene (E2-Crimson gene, prepared by PCR using pE2-Crimson (Clontech Laboratories, Inc.) as a template) downstream of a CMV promoter of pOL1.

The results are shown in FIG. 19. The EGFP-targeting artificial miRNA expressed by the pre-miR-367-based artificial miRNA precursor caused decrease of fluorescence intensity of EGFP by about 73% and was found to exhibit a high gene knockdown effect.

6. Target Gene Knockdown Effect of Artificial miRNA Expressed by Artificial miRNA Precursor Based on Pre-miR-367 (3)

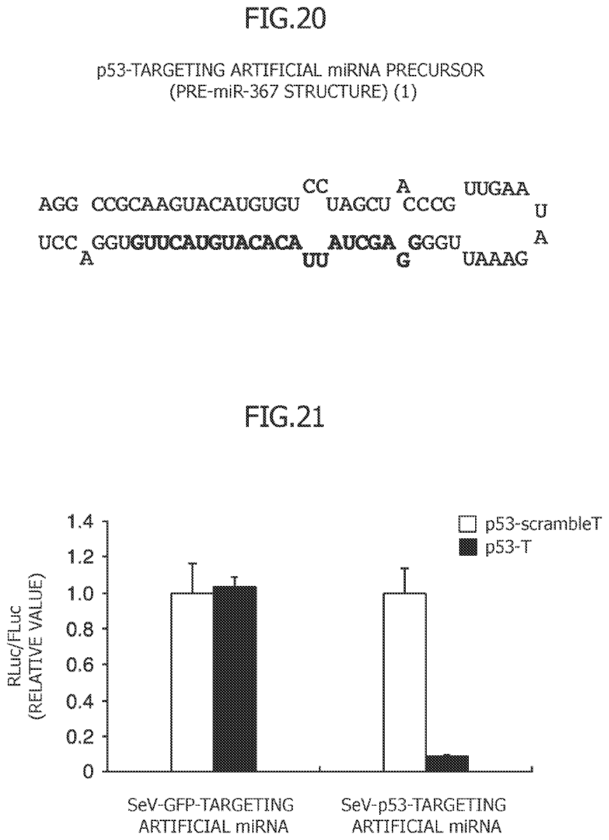

On the basis of the structure of pre-miR-367, an artificial miRNA precursor mimicking the secondary structure thereof and targeting mouse p53 was produced, and the gene knockdown effect of the mouse p53-targeting artificial miRNA expressed from a SeV vector incorporating the artificial miRNA precursor was evaluated. A sequence completely complementary to a target sequence in an mRNA for mouse p53, the sequence described by Dirac and Bernards (J. Biol. Chem., (2003), Vol. 278, No. 14, pp. 11731-11734), was used for the sequence of the artificial miRNA. The nucleotide sequence of the mouse p53-targeting artificial miRNA precursor is shown in Table 6, and the secondary structure is shown in FIG. 20. In the figure, the miRNA sequence targeting the mouse p53 gene is shown in bold.

TABLE 6
Nucleotide sequence of precursor

| Name of artificial miR | Nucleotide sequence | SEQ ID NO |
|---|---|---|
| p53-targeting artificial miRNA | AGGCCGCAAGUACAUGUGUCCUAGCUACCC | 28 |
| precursor (pre-miR-367 structure) (1) | GUUGAAUAGAAAUUGGGGAGCUAUUACACA | |
| | UGUACUUGUGGACCU | |

A SeV-p53-targeting artificial miRNA, being a SeV vector incorporating mouse p53-targeting miRNA precursor (1), a hygromycin resistance gene, and a Keima-Red gene, was prepared using the same procedure as in (1-1). The expression vector was introduced into HCT116 cells using the same procedure as in (1-2), the medium was supplemented with 100 μg/ml hygromycin B from the next day, and cells stably retaining the SeV vector genome were selected. Into the cells obtained, a reporter plasmid obtained by incorporating the mouse p53 target sequence into the 3′ untranslated region of the RLuc gene was introduced using the same procedure as in (1-3), and the gene knockdown effect was evaluated. The mouse p53 target sequence and the corresponding scramble sequence are shown in the following.

TABLE 7
p53 Target sequence and corresponding
scramble sequence

| Name of target | | SEQ |
|---|---|---|
| sequence | Nucleotide sequence | ID NO |
| p53-T | CAAGTACATGTGTAATAGCTCC | 29 |
| Name of scramble | | SEQ |
| sequence | Nucleotide sequence | ID NO |
| p53-scrambleT | GCCTAATATACGATGGCTATCA | 30 |

The results are shown in FIG. 21. The mouse p53-targeting artificial miRNA expressed from the mouse p53-targeting pre-miR-367-based artificial miRNA precursor caused the decrease of reporter RLuc activity by about 91%, and was found to exhibit a high target gene knockdown effect.

Furthermore, artificial miRNA precursors targeting different sites in the mouse p53 mRNA were produced. The nucleotide sequences of mouse p53-targeting artificial miRNA precursors (2) and (3) are shown in Table 8.

TABLE 8
Nucleotide sequences of precursors

| Name of artificial miR | Nucleotide sequence | SEQ ID NO |
|---|---|---|
| p53-targeting artificial miRNA | AGGCCGUGGGACAGCCAAGGAUGUUACGCU | 31 |
| precursor (pre-miR-367 structure) (2) | GUUGAAUAGAAAUUGGCAUAACAGACUUGG | |
| | CUGUCCCAUGGACCU | |
| p53-targeting artificial miRNA | AGGCCGCGGGUGGAAGGAACCUUGUAACCU | 32 |
| precursor (pre-miR-367 structure) (3) | GUUGAAUAGAAAUUGGGAUACAAAUUUCCU | |
| | UCCACCCGUGGACCU | |

SeV-p53-targeting artificial miRNA (1), SeV-p53-targeting artificial miRNA (2), and SeV-p53-targeting artificial miRNA (3), each being a SeV vector incorporating a mouse p53-targeting artificial miRNA precursor, a blasticidin resistance gene, and EGFP, were prepared using the same procedure as in (1-1). Each expression vector was introduced into HCT116 cells using the same procedure as in (1-2), the medium was supplemented with 10 μg/ml blasticidin from the next day, and cells stably retaining the SeV vector genome were selected. Into the cells obtained, a reporter plasmid obtained by incorporating the full-length open reading frame of mouse p53 into the 3′ untranslated region of the RLuc gene was introduced using the same procedure as in (1-3), and the gene knockdown effects were evaluated.

The results are shown in FIG. 24. All the p53-targeting artificial miRNAs efficiently suppressed reporter RLuc activity. These confirmed that the effect of artificial miRNA expressed from a SeV vector is not limited only to specific target sequences.

7. Production of iPS Cells Using SeV-p53-Targeting Artificial miRNA Precursor

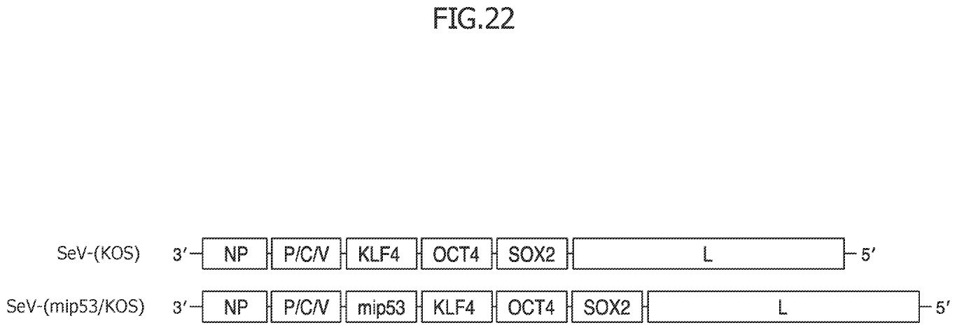

To produce iPS cells, c-MYC is typically introduced in addition to three reprogramming factors: KLF4, OCT4, and SOX2. However, c-MYC is an oncogene, and therefore, a problem arises of risk of promoting tumor formation. With regard to this, it has been reported that use of shRNA targeting p53 enables promotion of iPS cell induction (Nature, (2009), Vol. 460, No. 7259, pp. 1140-1144), and thus, whether iPS cells can be produced by expressing p53-targeting artificial miRNA from a SeV vector, instead of the c-MYC gene, was tested. A SeV vector incorporating a mouse p53-targeting artificial miRNA precursor, a KLF4 gene, an OCT4 gene, and a SOX2 gene was prepared in the same procedure as in (1-1). The KLF4 gene, OCT4 gene, and SOX2 gene had been obtained by artificial gene synthesis (GenScript Biotech). The genomic configurations of the SeV-(KOS) vector and SeV-(mip53/KOS) vector are shown in FIG. 22.

SeV-(KOS) and SeV-(mip53/KOS) were introduced into MEF at MOI=5. The next day, cells into which a vector had been introduced (1×10⁴ cells) were placed on MEF treated with mitomycin C and were cultured in mouse ES medium. On day 14, immunostaining was performed by using an antibody against SSEA1 (eBioscience), a pluripotency marker. Cells into which a SeV vector, into which no foreign gene had been introduced (SeV-empty), were used as a negative control.

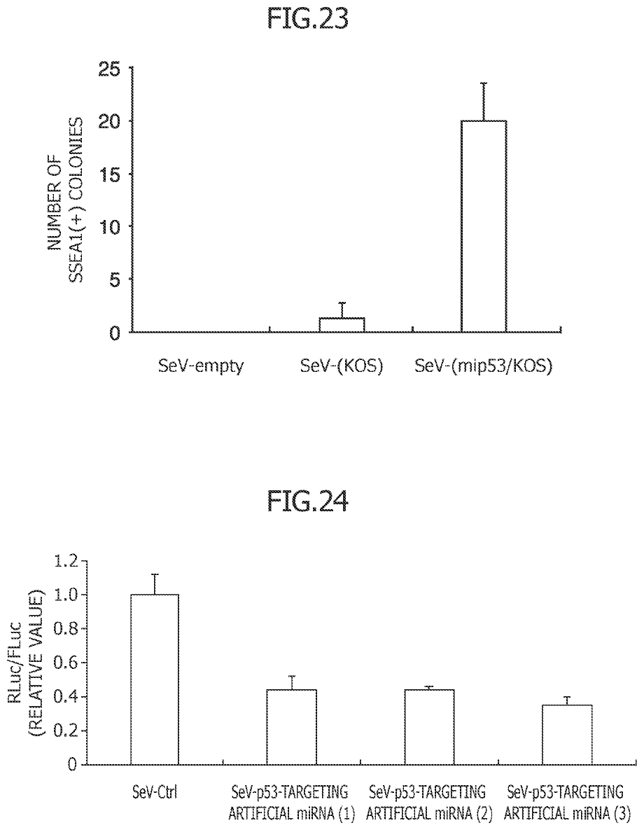

The results are shown in FIG. 23. It was demonstrated that expression of the p53-targeting artificial miRNA in addition to the three reprogramming factors KLF4, OCT4, and SOX2 promotes formation of SSEA1(+) colonies. This result suggested that the p53-targeting artificial miRNA precursor based on pre-miR-367 is useful for production of iPS cells.

1. A method for expressing natural or artificial miRNA, the method comprising:
introducing an isolated RNA molecule into a cell, the isolated RNA molecule comprising an artificial microRNA precursor comprising in the 5′→3′ direction:
a first terminal oligonucleotide;
a passenger strand oligonucleotide;
a first central oligonucleotide consisting of CYG (SEQ ID NO: 2), wherein Y is C or U;
a second central oligonucleotide consisting of a nucleotide sequence having at least 70% homology with UUGAAUAKAAAU (SEQ ID NO: 3), wherein K is G or U;
a third central oligonucleotide consisting of YGG (SEQ ID NO: 4), wherein Y is C or U;
a guide strand oligonucleotide comprising the natural or artificial miRNA; and
a second terminal oligonucleotide;
wherein the guide strand oligonucleotide consists of 17 to 29 nucleotides having complementarity to a target sequence in an mRNA of a target gene;
wherein the passenger strand oligonucleotide has a length identical to the length of the guide strand oligonucleotide or has a length one to three nucleotides shorter than the length of the guide strand oligonucleotide;
wherein the first terminal oligonucleotide consists of AGGCCR (SEQ ID NO: 1) or a nucleotide sequence in which one to three nucleotides in SEQ ID NO: 1 are substituted, wherein R is A or G;
wherein the second terminal oligonucleotide consists of UGGAYYK (SEQ ID NO: 5) or a nucleotide sequence in which one to three nucleotides in SEQ ID NO: 5 are substituted, wherein Y is C or U independently on each occurrence and K is G or U;
wherein the first terminal oligonucleotide and the second terminal oligonucleotide pair to form a first structural stem region;
wherein the passenger strand oligonucleotide and the guide strand oligonucleotide pair to form a double-stranded microRNA region;
wherein the first central oligonucleotide and the third central oligonucleotide pair to form a second structural stem region;
wherein the first structural stem region, the double-stranded microRNA region, and the second structural stem region together form a stem structure; and
wherein the second central oligonucleotide forms a loop structure.

2. The method according to claim 1, wherein the double-stranded microRNA region comprises a mismatch or a bulge.

3. The method according to claim 1, wherein the isolated RNA molecule further comprises a spacer oligonucleotide consisting of 1 to 10 nucleotides between the first central oligonucleotide and the second central oligonucleotide or between the second central oligonucleotide and the third central oligonucleotide.

4. The method according to claim 1, wherein the isolated RNA molecule is introduced into a cell by an expression vector comprising the isolated RNA molecule or an RNA molecule consisting of a complementary sequence thereto, or a DNA molecule coding therefor.

5. The method according to claim 4, wherein the expression vector is an RNA virus vector.

6. The method according to claim 5, wherein the expression vector is a cytoplasmic RNA virus vector.

7. The method according to claim 6, wherein the expression vector is a Sendai virus vector.


SEQUENCE LISTING (33 sequences)

SEQ ID NO: 1 - RNA, 6 nt, Artificial Sequence
  1st terminal oligonucleotide
aggccr

SEQ ID NO: 2 - RNA, 3 nt, Artificial Sequence
  1st central oligonucleotide
cyg

SEQ ID NO: 3 - RNA, 12 nt, Artificial Sequence
  2nd central oligonucleotide
uugaauakaaau

SEQ ID NO: 4 - RNA, 3 nt, Artificial Sequence
  3rd central oligonucleotide
ygg

SEQ ID NO: 5 - RNA, 7 nt, Artificial Sequence
  2nd terminal oligonucleotide
uggayyk

SEQ ID NO: 6 - RNA, 6 nt, Artificial Sequence
  mouse 1st terminal oligonucleotide
aggccg

SEQ ID NO: 7 - RNA, 6 nt, Artificial Sequence
  human 1st terminal oligonucleotide
aggcca

SEQ ID NO: 8 - RNA, 7 nt, Artificial Sequence
  mouse 2nd terminal oligonucleotide
uggaccu

SEQ ID NO: 9 - RNA, 7 nt, Artificial Sequence
  human 2nd terminal oligonucleotide
uggauug

SEQ ID NO: 10 - RNA, 3 nt, Artificial Sequence
  mouse/human 1st central oligonucleotide
cug

SEQ ID NO: 11 - RNA, 3 nt, Artificial Sequence
  mouse/human 3rd central oligonucleotide
ugg

SEQ ID NO: 12 - RNA, 12 nt, Artificial Sequence
  mouse 2nd central oligonucleotide
uugaauagaaau

SEQ ID NO: 13 - RNA, 12 nt, Artificial Sequence
  human 2nd central oligonucleotide
uugaauauaaau

SEQ ID NO: 14 - DNA, 20 bp, Artificial Sequence
  124-T
ggcattcaccgcgtgcctta

SEQ ID NO: 15 - DNA, 23 bp, Artificial Sequence
  302a-T
tcaccaaaacatggaagcactta

SEQ ID NO: 16 - DNA, 22 bp, Artificial Sequence
  367-T
tcaccattgctaaagtgcaatt

SEQ ID NO: 17 - DNA, 20 bp, Artificial Sequence
  124-scrambleT
gcgaggttcccctgtctaca

SEQ ID NO: 18 - DNA, 23 bp, Artificial Sequence
  302a-scrambleT
gcagtacaccacgtaaatcaaat

SEQ ID NO: 19 - DNA, 22 bp, Artificial Sequence
  367-scrambleT
gcattcataattctcggaacta

SEQ ID NO: 20 - RNA, 75 nt, Artificial Sequence
  artificial miR-124 precursor (1)
aggccguuggcauucaccguaugccucacuguugaauagaaauugguaaggcacgcggugaaugccaauggaccu

SEQ ID NO: 21 - RNA, 75 nt, Artificial Sequence
  artificial miR-124 precursor (2)
aggccguuggcauucaccgcgugccuuacuguugaauagaaauugguaaggcacgcggugaaugccaauggaccu

SEQ ID NO: 22 - RNA, 75 nt, Artificial Sequence
  artificial miR targeting FLuc (367)
aggccgucacuuacgcugaucacuucuacuguugaauagaaauuggucgaaguacucagcguaagugauggaccu

SEQ ID NO: 23 - RNA, 109 nt, Artificial Sequence
  artificial miR targeting FLuc (124)
aucaagaucagagacucugcucuaccuuacgcuaagucauucggauuuaaugucauacaauucgaaguacucagcguaagugagagcggagccuacggcugcacuugaa

SEQ ID NO: 24 - RNA, 69 nt, Artificial Sequence
  artificial miR targeting FLuc (30)
gcgccacuuacgcugaguacuucgauagugaagccacagauguaucgaaguacucagcguaagugaugc

SEQ ID NO: 25 - RNA, 65 nt, Artificial Sequence
  artificial miR targeting FLuc (155)
cugucgaaguacucagcguaagugauuuuggccucugacugaucacuuuccgaguacuuugacag

SEQ ID NO: 26 - RNA, 69 nt, Artificial Sequence
  artificial miR targeting FLuc (302a)
ccacucaccuacgcugaucacuuuggcuuuagaccuaagaaagucgaaguacucagcguaagugauugg

SEQ ID NO: 27 - RNA, 75 nt, Artificial Sequence
  artificial miR targeting EGFP (367)
aggccgagcgcaccaucuuacucaaguacuguugaauagaaauugguccuugaagaagauggugcgcuuggaccu

SEQ ID NO: 28 - RNA, 75 nt, Artificial Sequence
  artificial miR targeting p53 (367) (1)
aggccgcaaguacauguguccuagcuacccguugaauagaaauuggggagcuauuacacauguacuuguggaccu

SEQ ID NO: 29 - DNA, 22 bp, Artificial Sequence
  p53-T
caagtacatgtgtaatagctcc

SEQ ID NO: 30 - DNA, 22 bp, Artificial Sequence
  p53-scrambleT
gcctaatatacgatggctatca

SEQ ID NO: 31 - RNA, 75 nt, Artificial Sequence
  artificial miR targeting p53 (367) (2)
aggccgugggacagccaaggauguuacgcuguugaauagaaauuggcauaacagacuuggcugucccauggaccu

SEQ ID NO: 32 - RNA, 75 nt, Artificial Sequence
  artificial miR targeting p53 (367) (3)
aggccgcggguggaaggaaccuuguaaccuguugaauagaaauugggauacaaauuuccuuccacccguggaccu

SEQ ID NO: 33 - RNA, 75 nt, Artificial Sequence
  miR-367 stem-loop structure fig. 9 sequence
aggccgucacuguugcuaacaugcaacucuguugaauagaaauuggaauugcacuuuagcaauggugauggaccu